(19) United States
(12) Patent Application Publication
(10) Pub. No.: US 2021/0330624 A1
(43) Pub. Date: Oct. 28, 2021

(54) COMPOSITIONS AND METHODS FOR TREATING OR PREVENTING DISEASES AND/OR DISORDERS CAUSED BY EXPOSURE TO AIR POLLUTION

(71) Applicant: Amarin Pharmaceuticals Ireland Limited, Dublin (IE)

(72) Inventors: Craig Granowitz, Bridgewater, NJ (US); Sephy Philip, Bridgewater, NJ (US)

(21) Appl. No.: 17/280,092

(22) PCT Filed: Sep. 25, 2019
(86) PCT No.: PCT/IB2019/001059

§ 371 Date: Mar. 25, 2021

Related U.S. Application Data
(60) Provisional application No. 62/736,897, filed on Sep. 26, 2018.

(51) Int. Cl. A61K 31/202; A61P 9/00

(52) U.S. Cl. CPC A61K 31/202; A61P 9/00

(57) ABSTRACT

In various embodiments, the present disclosure provides compositions and methods for treating and/or preventing diseases and disorders caused by exposure to air pollution and/or inhalation of particulate matter, such as oxidative stress, endothelial dysfunction, narrowing and/or thickening of arteries, and/or inflammation.

### PRIORITY CLAIM

This application claims priority to U.S. Provisional Patent Application Ser. No. 62/736,897 filed Sep. 26, 2018, the entire contents of which is incorporated herein by reference.

### BACKGROUND

Cardiovascular disease is one of the leading causes of death in the United States and most European countries. It is estimated that over 70 million people in the United States alone suffer from a cardiovascular disease or disorder, including but not limited to high blood pressure, coronary heart disease, dyslipidemia, congestive heart failure and stroke.

Long and/or short term exposure to air pollution contributes to the pathogenesis of cardiovascular diseases and disorders. It is approximated that air pollution causes seven million deaths per year worldwide with over half of the deaths attributable to the progression or development of a cardiovascular disease or disorder.

### SUMMARY

In various embodiments, methods of treating or preventing oxidative stress, endothelial dysfunction, narrowing and/or thickening of arteries, and/or inflammation induced by inhalation of particulate matter in a subject are provided. In one embodiment, the method comprises administering to the subject a composition comprising eicosapentaenoic acid or a derivative thereof, for example icosapent ethyl (“E-EPA”).

In another embodiment, methods of treating or preventing oxidative stress, endothelial dysfunction, narrowing and/or thickening of arteries, and/or inflammation induced by long term and/or short term exposure to air pollution in a subject are provided. In one such embodiment, the method comprises administering to the subject a composition comprising eicosapentaenoic acid or a derivative thereof such as E-EPA.

In another embodiment, methods of treating or preventing oxidative stress, endothelial dysfunction, narrowing and/or thickening of arteries, and/or inflammation induced by inhalation of particulate matter are provided. In one such embodiment, the method comprises administering to the subject a composition comprising eicosapentaenoic acid or a derivative thereof such as E-EPA, wherein administration of the composition reduces a risk of atherosclerotic cardiovascular disease in the subject.

These and other embodiments of the present invention will be disclosed in further detail herein below.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

In one embodiment, the disclosure provides a method for treatment and/or prevention of cardiovascular-related diseases. The term “cardiovascular-related disease” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

In one embodiment, the disclosure provides a method for treatment and/or prevention of oxidative stress, endothelial dysfunction, narrowing and/or thickening of arteries, and/or inflammation. In another embodiment, the disclosure provides a method of treatment and/or prevention of diseases associated with oxidative stress, endothelial dysfunction, narrowing and/or thickening of arteries, and/or inflammation caused by the exposure to air pollution and/or inhalation of particulate matter. The term “oxidative stress” herein refers to the increased formation of reactive oxygenated species (ROS) and/or decreased antioxidative potential (i.e., capacity to reduce or impair the generation of ROS) in an afflicted person. The term “endothelial dysfunction” herein refers to damage or degradation of the endothelial lining caused by numerous factors including but not limited to, high blood pressure, high blood glucose levels, and/or increased blood lipid levels. Endothelial dysfunction can then lead to reduced function in endothelium-dependent vasodilation, procoagulation, and proinflammatory response. The term “narrowing” of arteries herein refers to a condition characterized by a decreased or a complete reduction in blood flow and oxygen transport to target tissues and organs of an afflicted person that occurs for example, from the formation of plaque within the arterial wall and/or as a result of inflammation causing a swelling of the arterial wall. An occlusion (i.e., blockage) of the arteries prevents sufficient blood flow and thereby, oxygen transport to target tissues and organs, which can lead to a wide range of illness such as, but not limited to, hypoxia, myocardial infraction, stroke, and/or pulmonary embolism. The term “thickening” of arteries herein refers to an actual thickening of the arterial wall (i.e., an increase in the ratio of the wall thickness-to-radius of the artery) and/or an actual enlargement of the arterial wall (i.e., dilatation). This thickening of the arterial wall can lead to a weakened and narrowed arterial wall, which overtime, can cause irregular blood flow and oxygen transport. On some occasions, the thickening of the arterial wall can result in an actual rupture of the wall, preventing blood flow and oxygen transport. Both a partial and complete block in blood flow and oxygen transport to target tissues can result in subsequent organ and tissue damage and/or death. The narrowing and thickening of the arterial wall can occur independently or dependently of each other. The term “inflammation” herein refers to pulmonary and/or systemic inflammation. Pulmonary inflammation is characterized by inflammation of the pulmonary system, resulting in restricted oxygen flow due to a narrowing of the air passageways of an afflicted person. The term “pulmonary system” herein refers to those organs and/or structures responsible for taking in oxygen into and/or expelling carbon dioxide from the body. The organs and/or structures include, but are not limited to, those associated with the nasal, pharyngeal and laryngeal passageways, the trachea, bronchi, bronchioles, and/or alveoli. In one embodiment, the alveoli in the lungs becomes inflamed, which can decrease the follow of oxygen through the alveoli to the bloodstream. This narrowing of the air passageways causes episodic dyspnea, coughing, and/or wheezing, all of which are associated with asthma, and in severe cases, causes death. Systemic inflammation is characterized by the widespread inflammation throughout the body of an afflicted person. Systemic inflammation leads to the degradation of both the structure and function of essential organs, such as the muscle, heart, and liver, compromises the immune system, and also causes multi-organ failure and death.

In another embodiment, the disclosure provides a method for treatment and/or prevention of oxidative stress, endothelial dysfunction, narrowing and/or thickening of arteries, and/or inflammation in a subject induced by long term or short term exposure to air pollution. The term “long term” in the present context refers to exposure to air pollution for a period of time greater than or equal to one year. There term “short term” herein refers to exposure to air pollution for a period of time less than one year.

In another embodiment, the disclosure provides a method for reducing the risk of an atherosclerotic cardiovascular disease. The term “atherosclerotic cardiovascular disease” herein refers to any condition characterized by plaque accumulation on vessel walls and vascular inflammation.

In another embodiment, the disclosure provides a method of suppressing an inflammatory response caused by the inhalation of particulate matter. The inflammatory response is observed in not only the lungs, but also other organs, to included but not limited to the brain, liver, kidneys, and spleen.

In another embodiment, the disclosure provides a method of treatment and/or prevention of oxidative stress, endothelial dysfunction, narrowing and/or thickening of arteries, and/or inflammation induced by the inhalation of particulate matter. The term “particulate matter” herein refers to a mixture of species generated from numerous emission sources. The particulate matter may be emitted directly into the air in the form of soot, smoke, and/or dust. The particulate matter can be formed in the atmosphere from the reactions of gases including by not limited to nitric oxides (NOₓ), sulfur oxides (SOₓ), reactive organic gases (ROG), and/or ammonia. In one embodiment, the polluted air contains particulate matter. In one embodiment, the particulate matter is a mixture of particulates of varying sizes. The various sizes of particulate matter are classified as coarse, fine, and ultrafine. In some embodiments, coarse particulate matter refers to particulates that have a diameter that are less than, or particulates having a mean or median diameter, on a volume basis, less than about 10 μm but greater than about 2.5 μm in diameter (PM_{2.5-10}). In some embodiments, coarse particulate matter refers to particulates that have a diameter less than, or particulates having a mean or median diameter, on a volume basis, less than about 9 μm, less than about 8 μm, less than about 7 μm, less than about 6 μm, less than about 5 μm, less than about 4 μm, and/or less than about 3 μm. In some embodiments, fine particulate matter refers to particulates of about, or particulates having a mean or median diameter, on a volume basis, of about 2.5 μm in diameter (PM_{2.5}). In some embodiments, ultrafine particulate matter refers to particulates that are less than, or particulates having a mean or median diameter, on a volume basis, less than about 0.1 in diameter (PM_{0.1}). In some embodiments, ultrafine particulate matter refers to particulates that are less than, or particulates having a mean or median diameter, on a volume basis, less than about 0.05 μm, less than about 0.02 μm, and/or less than about 0.01 μm.

The term “treatment” in relation to a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing, or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In another embodiment of the disclosure, the term “treating” refers to a method of initiating therapy after exposure to air pollution. In another aspect of the disclosure, the term “preventing” refers to a method of initiating therapy prior to exposure to air pollution.

In one embodiment, the present disclosure provides a method of blood lipid therapy comprising administering to a subject or subject group in need thereof a pharmaceutical composition as described herein. In another embodiment, the subject or subject group has hypertriglyceridemia, hypercholesterolemia, mixed dyslipidemia, and/or very high triglycerides.

In another embodiment, the subject or subject group being treated has a baseline triglyceride level (or mean or median baseline triglyceride level in the case of a subject group), fed or fasting, of about 200 mg/dL to about 500 mg/dL. In another embodiment, the subject or subject group has a baseline LDL-C level (or mean or median baseline LDL-C level), despite stable statin therapy, of about 40 mg/dL to about 115 or about 40 to about 100 mg/dL.

In one embodiment, the subject or subject group being treated in accordance with methods of the disclosure is on concomitant statin therapy, for example atorvastatin, rosuvastatin, or simvastatin therapy (with or without ezetimibe). In another embodiment, the subject is on concomitant stable statin therapy at time of initiation of ultra-pure EPA therapy.

In another embodiment, the subject or subject group being treated in accordance with methods of the disclosure has a body mass index (BMI or mean BMI) of not more than about 45 kg/m².

In one embodiment, the disclosure provides a method of lowering triglycerides in a subject on stable statin therapy having baseline fasting triglycerides of about 200 mg/dL to about 500 mg/dL, the method comprising administering to the subject a pharmaceutical composition comprising about 1 g to about 4 g of EPA (e.g. ultra-pure EPA), wherein upon administering the composition to the subject daily for a period of about 12 weeks the subject exhibits at least 10%, at least 15%, at least 20%, at least 25%, at least 30%, at least 35%, at least 40%, at least 45%, at least 50%, at least 55%, at least 60%, at least 65%, at least 70%, or at least 75% lower fasting triglycerides than a control subject maintained on stable statin therapy (and optionally placebo matching the ultra-pure EPA) without concomitant ultra-pure EPA for a period of about 12 weeks, wherein the control subject also has baseline fasting triglycerides of about 200 mg/dL to about 500 mg/dL. The term “stable statin therapy” herein means that the subject, subject group, control subject, or control subject group in question has been taking a stable daily dose of a statin (e.g. atorvastatin, rosuvastatin or simvastatin) for at least 4 weeks prior to the baseline fasting triglyceride measurement (the “qualifying period”). For example, a subject or control subject on stable statin therapy would receive a constant daily (i.e. the same dose each day) statin dose for at least 4 weeks immediately prior to baseline fasting triglyceride measurement. In one embodiment, the subject's and control subject's LDL-C is maintained between about 40 mg/dL and about 115 mg/dL or about 40 mg/dL to about 100 mg/dL during the qualifying period. The subject and control subject are then continued on their stable statin dose for the 12 week period post baseline.

In one embodiment, the statin is administered to the subject and the control subject in an amount of about 1 mg to about 500 mg, about 5 mg to about 200 mg, or about 10 mg to about 100 mg, for example about 1 mg, about 2 mg, about 3 mg, about 4 mg, about 5 mg, about 6 mg, about 7 mg, about 8 mg, about 9 mg, or about 10 mg; about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In another embodiment, the subject (and optionally the control subject) has a baseline LDL-C level, despite stable statin therapy, of about 40 mg/dL to about 115 mg/dL or about 40 mg/dL to about 100 mg/dL. In another embodiment, the subject and/or control subject has a body mass index (BMI or mean BMI) of not more than about 45 kg/m².

In another embodiment, the disclosure provides a method of lowering triglycerides in a subject group on stable statin therapy having mean baseline fasting triglycerides of about 200 mg/dL to about 500 mg/dL, the method comprising administering to members of the subject group a pharmaceutical composition comprising about 1 g to about 4 g of ultra-pure EPA per day, wherein upon administering the composition to the members of the subject group daily for a period of about 12 weeks the subject group exhibits at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides than a control subject group maintained on stable statin therapy without concomitant ultra-pure EPA (optionally with matching placebo) for a period of about 12 weeks, wherein the control subject group also has mean baseline fasting triglycerides of about 200 mg/dL to about 500 mg/dL. In a related embodiment, the stable statin therapy will be sufficient such that the subject group has a mean LDL-C level at least about 40 mg/dL and not more than about 100 mg/dL or about 40 mg/dL to about 100 mg/dL for the 4 weeks immediately prior to the baseline fasting triglyceride measurement.

In another embodiment, the disclosure provides a method of lowering triglycerides in a subject group on stable statin therapy and having a mean baseline fasting triglyceride level of about 200 mg/dL to about 500 mg/dL, the method comprising administering to members of the subject group a pharmaceutical composition comprising about 1 g to about 4 g of ultra-pure EPA, wherein upon administering the composition to members of the subject group daily for a period of about 12 weeks the subject group exhibits: (a) at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides by comparison with a control subject group maintained on stable statin therapy without concomitant ultra-pure EPA (optionally with matching placebo) for a period of about 12 weeks, and (b) no serum LDL-C increase, no statistically significant serum LDL-C increase, a serum LDL-C decrease, or the subject is statistically non-inferior to the control subjects (statin plus optional placebo) in regard to serum LDL-C elevation) no increase in mean serum LDL-C levels compared to baseline, wherein the control subject also has mean baseline fasting triglycerides of about 200 mg/dL to about 500 mg/dL.

In another embodiment, the disclosure provides a method of lowering triglycerides in a subject on stable statin therapy and having a mean baseline fasting triglyceride level of about 200 mg/dL to about 500 mg/dL, the method comprising administering to the subject a pharmaceutical composition comprising about 1 g to about 4 g of ultra-pure EPA, wherein upon administering the composition to the subject daily for a period of about 12 weeks the subject exhibits (a) at least 10%, at least 15%, at least 20%, at least 25%, at least 30%, at least 35%, at least 40%, at least 45%, at least 50%, at least 55%, at least 60%, at least 65%, at least 70%, or at least 75% lower fasting triglycerides by comparison with a control subject maintained on stable statin therapy without concomitant ultra-pure EPA for a period of about 12 weeks and (b) no increase in serum LDL-C levels compared to baseline, wherein the control subject also has baseline fasting triglycerides of about 200 mg/dL to about 500 mg/dL.

In another embodiment, the disclosure provides a method of lowering triglycerides in a subject group on stable statin therapy and having a mean baseline fasting triglyceride level of about 200 mg/dL to about 500 mg/dL, the method comprising administering to members of the subject group a pharmaceutical composition comprising about 1 g to about 4 g of ultra-pure EPA, wherein upon administering the composition to the members of the subject group daily for a period of about 12 weeks the subject group exhibits: (a) at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides and (b) at least 5%, at least 10%, at least 15%, at least 20%, at least 25%, at least 30%, at least 35%, at least 40%, at least 45%, or at least 50% lower mean serum LDL-C levels by comparison with a control subject group maintained on stable statin therapy without concomitant ultra-pure EPA (optionally with matching placebo) for a period of about 12 weeks, no serum LDL-C increase, no statistically significant serum LDL-C increase, no statistically significant serum LDL-C increase, a serum LDL-C decrease, or the subject group is statistically non-inferior to the control subject group (statin plus optional placebo) in regard to serum LDL-C elevation), wherein the control subject group also has mean baseline fasting triglycerides of about 200 mg/dL to about 500 mg/dL.

In another embodiment, the disclosure provides a method of lowering triglycerides in a subject group on stable statin therapy and having a mean baseline fasting triglyceride level of about 200 mg/dL to about 500 mg/dL, the method comprising administering to members of the subject group a pharmaceutical composition comprising about 1 g to about 4 g of ultra-pure EPA, wherein upon administering the composition to the members of the subject group daily for a period of about 12 weeks the subject group exhibits (a) at least 10%, at least 15%, at least 20%, at least 25%, at least 30%, at least 35%, at least 40%, at least 45%, at least 50%, at least 55%, at least 60%, at least 65%, at least 70%, or at least 75% lower mean fasting triglycerides and (b) at least 5%, at least 10%, at least 15%, at least 20%, at least 25%, at least 30%, at least 35%, at least 40%, at least 45%, or at least 50% lower mean serum LDL-C levels by comparison with a control subject group maintained on stable statin therapy without concomitant ultra-pure EPA (optionally with matching placebo) for a period of about 12 weeks, no serum LDL-C increase, no statistically significant serum LDL-C increase, no statistically significant serum LDL-C increase, a serum LDL-C decrease, or the subject group is statistically non-inferior to the control subject group (statin plus optional placebo) in regard to serum LDL-C elevation), wherein the control subject group also has mean baseline fasting triglycerides of about 200 mg/dL to about 500 mg/dL.

In another embodiment, the disclosure provides methods for treating and/or preventing oxidative stress, endothelial dysfunction, narrowing and/or thickening of arteries, and/or inflammation in a subject caused by long term and/or short term exposure to air pollution and/or induced by inhaled particulate matter, the method comprising administering to the subject a pharmaceutical composition comprising about 1 g to about 4 g of ultra-pure EPA, where the subject further exhibits a reduction in triglycerides. In one such embodiment, there is no correlation or causality between the reduction in triglycerides and the prevention and/or treatment of oxidative stress, endothelial dysfunction, narrowing and/or thickening of arteries, and/or inflammation caused by long term and/or short term exposure to air pollution and/or induced by inhaled particulate matter.

In another embodiment, the subject or subject group being treated in accordance with methods of the disclosure exhibits a fasting baseline absolute plasma level of free total fatty acid (or mean thereof) not greater than about 300 nmol/ml, not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the disclosure exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the disclosure exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2%, or not more than about 0.15%. In one such embodiment, free plasma EPA and/or total fatty acid levels are determined prior to initiating therapy.

In another embodiment, the subject or subject group being treated in accordance with methods of the disclosure exhibits a fasting baseline absolute plasma level of free EPA (or mean thereof) not greater than about 1 nmol/ml, not greater than about 0.75 nmol/ml, not greater than about 0.50 nmol/ml, not greater than about 0.4 nmol/ml, not greater than about 0.35 nmol/ml, or not greater than about 0.30 nmol/ml.

In another embodiment, the subject or subject group being treated in accordance with methods of the disclosure exhibits a fasting baseline plasma, serum, or red blood cell (RBC) membrane EPA level not greater than about 150 μg/ml, not greater than about 125 μg/ml, not greater than about 100 μg/ml, not greater than about 95 μg/ml, not greater than about 75 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present disclosure comprise a step of measuring the subject's (or subject group's mean) baseline lipid profile prior to initiating therapy. In another embodiment, methods of the disclosure comprise the step of identifying a subject or subject group having one or more of the following: baseline non-HDL-C value (or mean) of about 200 mg/dL to about 400 mg/dL, for example at least about 210 mg/dL, at least about 220 mg/dL, at least about 230 mg/dL, at least about 240 mg/dL, at least about 250 mg/dL, at least about 260 mg/dL, at least about 270 mg/dL, at least about 280 mg/dL, at least about 290 mg/dL, or at least about 300 mg/dL; baseline total cholesterol value (or mean) of about 250 mg/dL to about 400 mg/dL, for example at least about 260 mg/dL, at least about 270 mg/dL, at least about 280 mg/dL or at least about 290 mg/dL; baseline VLDL-C value (or mean) of about 140 mg/dL to about 200 mg/dL, for example at least about 150 mg/dL, at least about 160 mg/dL, at least about 170 mg/dL, at least about 180 mg/dL, or at least about 190 mg/dL; baseline HDL-C value (or mean) of about 10 to about 100 mg/dL, for example not more than about 90 mg/dl not, not more than about 80 mg/dL, not more than about 70 mg/dL, not more than about 60 mg/dL, not more than about 60 mg/dL, not more than about 50 mg/dL, not more than about 40 mg/dL, not more than about 35 mg/dL, not more than about 30 mg/dL, not more than about 25 mg/dL, not more than about 20 mg/dL, or not more than about 15 mg/dL; and/or baseline LDL-C value (or mean) of about 30 to about 300 mg/dL, for example not less than about 40 mg/dL, not less than about 50 mg/dL, not less than about 60 mg/dL, not less than about 70 mg/dL, not less than about 90 mg/dL, or not less than about 90 mg/dL.

In another embodiment, methods of the present disclosure comprise treating and/or preventing oxidative stress, endothelial dysfunction, narrowing and/or thickening of arteries, and/or inflammation in a subject caused by long term and/or short term exposure to air pollution and/or induced by inhaled particulate matter. In another embodiment, methods of the disclosure comprise the step of identifying a subject or subject group having one or more of the following: baseline fasting triglyceride value (or mean) of at least about 150 mg/dL or less than about 150 mg/dL; baseline non-fasting triglyceride value (or mean) of at least about 150 mg/dL or less than about 150 mg/dL; baseline HDL-C value (or mean) of about 10 to about 100 mg/dL, for example not more than about 90 mg/dl not, not more than about 80 mg/dL, not more than about 70 mg/dL, not more than about 60 mg/dL, not more than about 60 mg/dL, not more than about 50 mg/dL, not more than about 40 mg/dL, not more than about 35 mg/dL, not more than about 30 mg/dL, not more than about 25 mg/dL, not more than about 20 mg/dL, or not more than about 15 mg/dL; and/or baseline LDL-C value (or mean) of about 30 to about 300 mg/dL, for example not less than about 40 mg/dL, not less than about 50 mg/dL, not less than about 60 mg/dL, not less than about 70 mg/dL, not less than about 90 mg/dL, or not less than about 90 mg/dL. In some embodiments, the subject or subjects are at least about forty-five (45) years of age, have diabetes and/or a CV disease, such as an atherosclerotic CV disease, and are on statin therapy, such as stable or concomitant statin therapy, or have otherwise been treated with a statin. In some embodiments, the subject or subjects have not been exposed to air pollution prior to initiating therapy.

In another embodiment, the present disclosure provides methods of treating and preventing oxidative stress, endothelial dysfunction, narrowing and/or thickening of arteries, and/or inflammation caused by exposure to air pollution and/or inhaled particulate matter. In some embodiments, the present disclosure provides a method of reducing cardiovascular risk in patients having fasting baseline triglycerides of about 200 mg/dL to about 500 mg/dL, LDL-C control, and LDL-C levels of about 40 mg/dL to 100 mg/dL, the method comprising administering to the subject a pharmaceutical composition comprising 4 g/day E-EPA, wherein upon administering the composition to the subject for a period of 12 weeks, the subject exhibits a reduction in triglycerides without raising LDL-C levels and significantly improved atherogenic and inflammatory parameters compared to baseline or placebo control. In some embodiments, the atherogenic and inflammatory parameters include non-high dense lipoprotein cholesterol (non-HDL-C), total cholesterol (TC), very low dense lipoprotein cholesterol (VLDL-C), lipoprotein-associated phospholipase A₂ (Lp-PLA₂), apolipoprotein B (Apo B), apolipoprotein C-III (Apo C-III), high dense lipoprotein (HDL-C), remnant lipoprotein (RLP-C), or very low dense lipoprotein triglyceride (VLDL-TG). In some embodiments, the subject exhibits a reduction in blood pressure. In some embodiments, the subject exhibits a reduction in insulin resistance. In some embodiments, the subject exhibits an increase in EPA and/or plasma levels in RBCs compared to baseline or placebo control.

In another embodiment, the present disclosure provides methods of reducing cardiovascular risk in patients having oxidative stress, endothelial dysfunction, narrowed and/or thickened arteries, and/or inflammation caused by exposure to air pollution and/or inhaled particulate matter. In some embodiments, the subject exhibits improvements in one or more of inflammatory biomarkers, metabolic biomarkers, oxidative biomarkers, and changes in heart rhythm. In some embodiments, the inflammatory biomarkers include vascular endothelial growth factor (VEGF), tumor necrosis factor-α (TNF-α), monocyte chemoattractant protein-1 (MCP-1), interleukin-1β (IL-1β), interleukin-6 (IL-6), soluble intercellular adhesion molecule-1 (sICAM-1), soluble vascular cellular adhesion molecule-1 (sVCAM-1), high sensitivity reactive protein (hsCRP), Lp-PLA₂, and circulating monocytes. In some embodiments, the oxidative biomarkers include lipid oxidation, lipid peroxidation, lipid hydroperoxidation, malondialdehyde, prostaglandin-2α (PGF-2α), platelet-derived growth factor (PDGF), and antioxidant potential levels. In some embodiments, the metabolic biomarkers include TC, VLDL-C, low dense lipoprotein cholesterol (LDL-C), high dense lipoprotein cholesterol (HDL-C), non-HDL-C, Apo B, apolipoprotein A-1 (Apo A-1), HDL-C functionality, and homeostasis model assessment of insulin resistance (HOMA-IR) levels. In some embodiments, changes in heart rhythm are assessed by arrhythmia suppression, ventricular arrhythmia rate, heart rate variability (HRV), and heart rate levels.

In a related embodiment, upon treatment in accordance with the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week, the subject or subject group exhibits one or more of the following outcomes:

(a) a reduction in triglyceride (TG) levels compared to baseline or placebo control (e.g., a subject on stable statin plus placebo matching the EPA treatment group);

(b) substantially no change (e.g., increase), no statistically significant change, or an increase in non high density lipoprotein cholesterol (non-HDL-C) levels compared to baseline or placebo control;

(c) a reduction in non-HDL-C levels compared to baseline or placebo control;

(d) substantially no change (e.g., increase), no statistically significant change, or an increase in HDL-C levels compared to baseline or placebo control;

(e) substantially no change (e.g., no increase), no statistically significant change, no increase, or an increase in low density level cholesterol (LDL-C) levels compared to baseline or placebo control;

(f) a reduction in LDL-C levels compared to baseline or placebo control;

(g) a reduction in apolipoprotein B (Apo B) levels compared to baseline or placebo control;

(h) a reduction in very low-density lipoprotein cholesterol (VLDL-C) levels compared to baseline or placebo control;

(i) an increase in apolipoprotein A-I (Apo A-I) levels compared to baseline or placebo control;

(j) an increase in apo A-I/Apo B ratio compared to baseline or placebo control;

(k) a reduction in lipoprotein (a) levels compared to baseline or placebo control;

(l) a reduction in (1) mean LDL particle number, (2) total LDL particle number, and/or (3) LDL particle number compared to baseline or placebo control;

(m) an increase in (1) mean LDL particle size, (2) total LDL particle size, and/or (3) LDL particle size compared to baseline or placebo control;

(n) a reduction in remnant-like particle cholesterol (RLP-C) compared to baseline or placebo control;

(o) a reduction in oxidized LDL compared to baseline or placebo control;

(p) substantially no change, no statistically significant change, or a reduction in fasting plasma glucose (FPG) levels compared to baseline or placebo control;

(q) substantially no change, no statistically significant change, or a reduction in hemoglobin A_{1c} (HbA_{1c}) compared to baseline or placebo control;

(r) a reduction in lipoprotein associated phospholipase A₂ (Lp-PLA₂) compared to baseline or placebo control;

(s) a reduction in intracellular adhesion molecule-1 (ICAM-1) and/or soluble ICAM-1 compared to baseline or placebo control;

(t) a reduction in interleukin-6 (IL-6) compared to baseline or placebo control;

(u) a reduction in plasminogen activator inhibitor-1 (PAI-1) compared to baseline or placebo control;

(v) a reduction in high sensitivity C-reactive protein (hsCRP) compared to baseline or placebo control;

(w) an increase in serum, plasma and/or RBC EPA compared to baseline or placebo control;

(z) an increase in serum phospholipid and/or RBC membrane EPA compared to baseline or placebo control;

(y) a reduction in one or more of serum phospholipid and/or RBC content of docosahexaenoic acid (DHA), docosapentaenoic acid (DPA), arachidonic acid (AA), palmitic acid (PA), stearidonic acid (SA), or oleic acid (OA) compared to baseline or placebo control;

(z) an increase in one or more of serum phospholipid and/or RBC content of docosahexaenoic acid (DHA), docosapentaenoic acid (DPA), arachidonic acid (AA), palmitic acid (PA), stearidonic acid (SA), or oleic acid (OA) compared to baseline or placebo control;

(aa) a reduction in total cholesterol (TC) levels compared to baseline or placebo control;

(bb) no change, no statistically significant change, or a reduction in vascular endothelial growth factor (VEGF) levels compared to baseline or placebo control;

(cc) a reduction in very low-density lipoprotein triglycerides (VLDL-TG) levels compared to baseline or placebo control;

(dd) a reduction in apolipoprotein C-III (Apo C-III) levels compared to baseline or placebo control;

(ee) substantially no change, no statistically significant change, or a reduction in circulating monocyte levels compared to baseline or placebo control;

(ff) substantially no change, no statistically significant change, or a reduction in tumor necrosis factor-α (TNF-α) levels compared to baseline or placebo control;

(gg) substantially no change, no statistically significant change, or reduction in monocyte chemoattractant protein-1 (MCP-1) levels compared to baseline or placebo control;

(hh) substantially no change, no statistically significant change, or a reduction in homeostatic model assessment of insulin resistance (HOMA-IR) levels compared to baseline or placebo control;

(ii) substantially no change, no statistically significant change, or a reduction in interleukin-1β (IL-1β) levels compared to baseline or placebo control;

(jj) substantially no change, no statistically significant change, or a reduction in lipid oxidation compared to baseline or placebo control;

(kk) substantially no change, no statistically significant change, or a reduction in lipid hydroperoxidation levels compared to baseline or placebo control.

(ll) substantially no change, no statistically significant change, or a reduction in malondialdehyde levels compared to baseline or placebo control;

(mm) substantially no change, no statistically significant change, or a reduction in lipid peroxidation levels compared to baseline or placebo control;

(nn) substantially no change, no statistically significant change, or a in reduction platelet-derived growth factor (PDGF) levels compared to baseline or placebo control;

(oo) substantially no change, no statistically significant change, or a reduction in prostaglandin-F2α (PGF-2α) levels compared to baseline or placebo control;

(pp) substantially no change, no statistically significant change, or a reduction in soluble vascular cellular adhesion molecule-1 (sVCAM-1) levels compared to baseline or placebo control;

(qq) substantially no change, no statistically significant change, or an increase in arrhythmia suppression levels compared to baseline or placebo control;

(rr) substantially no change, no statistically significant change, or a reduction in ventricular arrhythmia rates compared to baseline or placebo control;

(ss) substantially no change, no statistically significant change, or an increase in heart rate variability (HRV) levels compared to baseline or placebo control;

(tt) substantially no change, no statistically significant change, or a reduction in heart rate levels compared to baseline or placebo control;

(uu) substantially no change, no statistically significant change, or a reduction in blood pressure compared to baseline or placebo control;

(vv) substantially no change, no statistically significant change, or an increase in antioxidant potential levels compared to baseline or placebo control; and/or

(ww) substantially no change (e.g., no increase), no statistically significant change, no increase, or an increase in HDL-C functionality compared to baseline or placebo control.

In one embodiment, methods of the present disclosure comprise measuring baseline levels of one or more markers set forth in (a)-(ww) above prior to dosing the subject or subject group. In another embodiment, the methods comprise administering a composition as disclosed herein to the subject after baseline levels of one or more markers set forth in (a)-(ww) are determined, and subsequently taking an additional measurement of said one or more markers.

In another embodiment, upon treatment with a composition of the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more, any 24 or more, or all 25 of outcomes (a)-(ww) described immediately above.

In another embodiment, upon treatment with a composition of the present disclosure, the subject or subject group exhibits one or more of the following outcomes:

(a) a reduction in TG level of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control (e.g., a subject on statin and placebo matching the EPA treatment group);

(b) substantially no change (e.g., no increase), no statistically significant change, or an increase in non-HDL-C levels of less than 30% increase, less than 20% increase, less than 10% increase, less than 5% increase (actual % change or median % change), or no increase in non-HDL-C levels as compared to baseline or placebo control;

(c) a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control;

(d) substantially no change, no change, or an increase in HDL-C levels of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control;

(e) substantially no change, no statistically significant change, no increase, or an increase in LDL-C levels by a less than 60% increase, less than 50% increase, less than 40% increase, less than 30% increase, less than 20% increase, less than 10% increase, less than 5% (actual % change or median % change) increase as compared to baseline or placebo control;

(f) a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55%, or at least about 75% (actual % change or median % change) as compared to baseline or placebo control;

(g) a reduction in Apo B levels of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control;

(h) a reduction in VLDL-C levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(i) an increase in Apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(j) an increase in apo A-I/Apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(k) a reduction in lipoprotein (a) levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(l) a reduction in (1) mean LDL particle number, (2) total LDL particle number, and/or (3) LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(m) an increase in (1) mean LDL particle size, (2) total LDL particle size, and/or (3) LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(n) a reduction in RLP-C of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(o) a reduction in oxidized LDL of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(p) substantially no change, no statistically significant change, or a reduction in FPG of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(q) substantially no change, no statistically significant change, a reduction in HbA_{1c} of at least about 5%, at least about 10%, at least about 15%, at least about 20%, at least about 25%, at least about 30%, at least about 35%, at least about 40%, at least about 45%, or at least about 50% (actual % change or median % change) compared to baseline or placebo control;

(r) a reduction in Lp-PLA₂ of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(s) a reduction in ICAM-1 and/or soluble ICAM-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(t) a reduction in IL-6 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(u) a reduction in PAI-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(v) a reduction in hsCRP of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(w) an increase in serum, plasma and/or RBC EPA of at least about 5%, at least about 10%, at least about 15%, at least about 20%, at least about 25%, at least about 30%, at least about 35%, at least about 40%, at least about 45%, at least about 50%, at least about 100%, at least about 200%, or at least about 400% (actual % change or median % change) compared to baseline or placebo control;

(x) an increase in serum phospholipid and/or RBC membrane EPA of at least about 5%, at least about 10%, at least about 15%, at least about 20%, at least about 25%, at least about 30%, at least about 35%, at least about 40%, at least about 45%, at least about 50%, at least about 100%, at least about 200%, or at least about 400% (actual % change or median % change) compared to baseline or placebo control;

(y) a reduction in one or more of serum phospholipid and/or RBC DHA, DPA, AA, SA, PA, and/or OA of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) compared to baseline or placebo control;

(z) an increase in one or more of serum phospholipid and/or RBC DHA, DPA, AA, SA, PA, and/or OA of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) compared to baseline or placebo control

(aa) a reduction in total cholesterol of at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) compared to baseline or placebo control;

(bb) no change, no statistically significant change, or a reduction in VEGF levels compared to baseline or placebo control 0.5%, at least about 1%, at least about 2%, at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(cc) a reduction in VLDL-TG of at least about 5%, at least about 10%, at least about 15%, at least about 20%, at least about 25%; or at least 30%, at least about 35%, at least about 40%, at least about 45%, at least about 50%, or at least about 100% (actual % change or median % change) compared to baseline or placebo control;

(dd) a reduction in Apo C-III of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control;

(ee) substantially no change, no statistically significant change, or a reduction in circulating monocyte of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control;

(ff) substantially no change, no statistically significant change, or reduction in TNF-α levels of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control;

(gg) substantially no change, no statistically significant change, or reduction in MCP-1 levels of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control; and

(hh) substantially no change, no statistically significant change, or a reduction in HOMA-IR levels of at least about 5%, at least about 10%, at least about 15%, at least about 20%, or at least about 25%; at least about 30%, at least about 35%, at least about 40%, at least about 45%, at least about 50%, or at least about 100% (actual % change or median % change) compared to baseline or placebo control.

(ii) substantially no change, no statistically significant change, or a reduction IL-1β levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(jj) substantially no change, no statistically significant change, or a reduction lipid oxidation levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(kk) substantially no change, no statistically significant change, or a reduction lipid hydroperoxide levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(ll) substantially no change, no statistically significant change, or a reduction malondialdehyde levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(mm) substantially no change, no statistically significant change, or a reduction lipid peroxide levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(nn) substantially no change, no statistically significant change, or a reduction PDGF levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(oo) substantially no change, no statistically significant change, or a reduction in PGF-2a levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(pp) substantially no change, no statistically significant change, or a reduction in sVCAM-1 levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(qq) substantially no change, no statistically significant change, or an increase in arrhythmia suppression levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(rr) substantially no change, no statistically significant change, or a reduction in ventricular arrhythmia rates of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(ss) substantially no change, no statistically significant change, or an increase in HRV levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(tt) substantially no change, no statistically significant change, or a reduction in heart rate levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(uu) substantially no change, no statistically significant change, or a reduction in blood pressure of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control;

(vv) substantially no change, no statistically significant change, or an increase in antioxidant potential levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control; and/or

(ww) substantially no change (e.g., no increase), no statistically significant change, no increase, or an increase in HDL-C functionality of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline or placebo control.

In one embodiment, methods of the present disclosure comprise measuring baseline levels of one or more markers set forth in (a)-(ww) prior to dosing the subject or subject group. In another embodiment, the methods comprise administering a composition as disclosed herein to the subject after baseline levels of one or more markers set forth in (a)-(ww) are determined, and subsequently taking a second measurement of the one or more markers as measured at baseline for comparison thereto.

In another embodiment, upon treatment with a composition of the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks, or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, or all 27 or more of outcomes (a)-(ww) described immediately above.

Parameters (a)-(ww) can be measured in accordance with any clinically acceptable methodology. For example, triglycerides, total cholesterol, HDL-C and fasting blood sugar can be sampled from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A-1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A₂ can be determined from EDTA plasma or serum and serum, respectively, using enzymatic immunoseparation techniques. Oxidized LDL, intercellular adhesion molecule-1 and interleukin-2 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company.

In one embodiment, subjects fast for up to 12 hours prior to blood sample collection, for example about 10 hours.

In another embodiment, the subject being treated is in the highest risk category of Adult Treatment Panel (ATP) III Classification of LDL, Total, and HDL Cholesterol (mg/dL) (e.g., CHD or CHD Risk Equivalents (10-year risk of more than about 20%)). In another embodiment, the subject is in the ATP III Multiple (2+) risk factor category.

In one embodiment, the disclosure provides a method of lowering triglycerides in a subject in the highest risk category of Adult Treatment Panel (ATP) III Classification of LDL, Total, and HDL Cholesterol (mg/dL) (e.g., CHD or CHD Risk Equivalents (10-year risk of more than about 20%)). In another embodiment, the subject is in the ATP III Multiple (2+) risk factor category. In another embodiment, the method includes a step of identifying a subject in the ATP III Multiple (2+) risk factor category prior to administering ultra-pure E-EPA to the subject.

In another embodiment, the present disclosure provides a method of treating or preventing primary hypercholesterolemia and/or mixed dyslipidemia (Fredrickson Types IIa and IIb) in a patient in need thereof, comprising administering to the patient one or more compositions as disclosed herein. In a related embodiment, the present disclosure provides a method of reducing triglyceride levels in a subject or subjects when treatment with a statin or niacin extended-release monotherapy is considered inadequate (Frederickson type IV hyperlipidemia).

In another embodiment, the present disclosure provides a method of treating or preventing risk of recurrent nonfatal myocardial infarction in a patient with a history of myocardial infarction, comprising administering to the patient one or more compositions as disclosed herein.

In another embodiment, the present disclosure provides a method of slowing progression of or promoting regression of atherosclerotic disease in a patient in need thereof, comprising administering to a subject in need thereof one or more compositions as disclosed herein.

In another embodiment, the present disclosure provides a method of treating or preventing very high serum triglyceride levels (e.g., Types IV and V hyperlipidemia) in a patient in need thereof, comprising administering to the patient one or more compositions as disclosed herein.

In one embodiment, a composition of the disclosure is administered to a subject in an amount sufficient to provide a daily dose of EPA of about 1 mg to about 10,000 mg, about 25 mg to about 5000 mg, about 50 mg to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, or about 4100 mg.

In another embodiment, any of the methods disclosed herein are used in treatment of a subject or subjects that consume a traditional Western diet. In one embodiment, the methods of the disclosure include a step of identifying a subject as a Western diet consumer or a prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60%, or more or 70% of total calories come from these sources.

In another embodiment, any of the methods disclosed herein are used in treatment of a subject or subjects that consume less than (actual or average) about 150 g, less than about 125 g, less than about 100 g, less than about 75 g, less than about 50 g, less than about 45 g, less than about 40 g, less than about 35 g, less than about 30 g, less than about 25 g, less than about 20 g, or less than about 15 g of fish per day.

In another embodiment, any of the methods disclosed herein are used in treatment of a subject or subjects that consume less than (actual or average) about 10 g, less than about 9 g, less than about 8 g, less than about 7 g, less than about 6 g, less than about 5 g, less than about 4 g, less than about 3 g, or less than about 2 g per day of omega-3 fatty acids from dietary sources.

In another embodiment, any of the methods disclosed herein are used in treatment of a subject or subjects that consume less than (actual or average) about 2.5 g, less than about 2 g, less than about 1.5 g, less than about 1 g, less than about 0.5 g, less than about 0.25 g, or less than about 0.2 g per day of EPA and DHA (combined) from dietary sources.

In one embodiment, compositions useful in various embodiments of the disclosure comprise a polyunsaturated fatty acid as an active ingredient. In another embodiment, such compositions comprise EPA as an active ingredient. The term “EPA” as used herein refers to eicosapentaenoic acid (e.g., eicosa-5,8,11,14,17-pentaenoic acid) and/or a pharmaceutically acceptable ester, derivative, conjugate, or salt thereof, or mixtures of any of the foregoing.

In one embodiment, the EPA comprises all-cis eicosa-5,8,11,14,17-pentaenoic acid. In another embodiment, the EPA is in the form of an eicosapentaenoic acid ester. In another embodiment, the EPA comprises a C₁-C₅ alkyl ester of EPA. In another embodiment, the EPA comprises eicosapentaenoic acid ethyl ester, eicosapentaenoic acid methyl ester, eicosapentaenoic acid propyl ester, or eicosapentaenoic acid butyl ester. In still another embodiment, the EPA comprises all-cis eicosa-5,8,11,14,17-pentaenoic acid ethyl ester.

In still other embodiments, the EPA comprises ethyl-EPA, lithium EPA, mono, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree.

The term “pharmaceutically acceptable” in the present context means that the substance in question does not produce unacceptable toxicity to the subject or interaction with other components of the composition.

In one embodiment, EPA present in a composition suitable for use according to the disclosure comprises ultra-pure EPA. The term “ultra-pure” as used herein with respect to EPA refers to a composition comprising at least 96% by weight EPA (as the term “EPA” is defined and exemplified herein). Ultra-pure EPA can comprise even higher purity EPA, for example at least 97% by weight EPA, at least 98% by weight EPA, or at least 99% by weight EPA, wherein the EPA is any form of EPA as set forth herein. Ultra-pure EPA can further be defined (e.g., impurity profile) by any of the description of EPA provided herein.

In some embodiments, EPA is present in a composition in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg.

In various embodiments, one or more antioxidants can be present in the EPA (e.g., E-EPA or ultra pure E-EPA). Non-limiting examples of suitable antioxidants include tocopherol, lecithin, citric acid, and/or ascorbic acid. One or more antioxidants, if desired, are typically present in the EPA in an amount of about 0.01% to about 0.1%, by weight, or about 0.025% to about 0.05%, by weight.

In one embodiment, a composition of the disclosure contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of total fatty acids, docosahexaenoic acid or derivative thereof such as E-DHA, if any. In another embodiment, a composition of the disclosure contains substantially no docosahexaenoic acid or derivative thereof such as E-DHA. In still another embodiment, a composition of the disclosure contains no docosahexaenoic acid or E-DHA.

In another embodiment, EPA represents at least about 60%, at least about 70%, at least about 80%, at least about 90%, at least about 95%, at least about 97%, at least about 98%, at least about 99%, or 100%, by weight, of all fatty acids present in a composition useful in accordance with the disclosure.

In another embodiment, a composition of the disclosure contains less than 30%, less than 20%, less than 10%, less than 9%, less than 8%, less than 7%, less than 6%, less than 5%, less than 4%, less than 3%, less than 2%, less than 1%, less than 0.5%, or less than 0.25%, by weight of the total composition or by weight of the total fatty acid content, of any fatty acid other than EPA, or derivative thereof. Illustrative examples of a “fatty acid other than EPA” include linolenic acid (LA) or derivative thereof such as ethyl-linolenic acid, arachidonic acid (AA) or derivative thereof such as ethyl-AA, docosahexaenoic acid (DHA) or derivative thereof such as ethyl-DHA, alpha-linolenic acid (ALA) or derivative thereof such as ethyl-ALA, stearadonic acid (STA) or derivative thereof such as ethyl-SA, eicosatrienoic acid (ETA) or derivative thereof such as ethyl-ETA, and/or docosapentaenoic acid (DPA) or derivative thereof such as ethyl-DPA.

In another embodiment, a composition of the disclosure has one or more of the following features: (a) eicosapentaenoic acid ethyl ester represents at least 96%, at least 97%, or at least 98%, by weight, of all fatty acids present in the composition; (b) the composition contains not more than 4%, not more than 3%, or not more than 2%, by weight, of total fatty acids other than eicosapentaenoic acid ethyl ester; (c) the composition contains not more than 0.6%, 0.5%, 0.4%, or 0.3% of any individual fatty acid other than eicosapentaenoic acid ethyl ester; (d) the composition has a refractive index (20 ° C.) of about 1 to about 2, about 1.2 to about 1.8, or about 1.4 to about 1.5; (e) the composition has a specific gravity (20 ° C.) of about 0.8 to about 1.0, about 0.85 to about 0.95, or about 0.9 to about 0.92; (f) the composition contains not more than 20 ppm, 15 ppm, or 10 ppm heavy metals, (g) the composition contains not more than 5 ppm, 4 ppm, 3 ppm, or 2 ppm arsenic, and/or (h) the composition has a peroxide value not more than 5, 4, 3, or 2 Meq/kg.

In another embodiment, a composition useful in accordance with the disclosure comprises, consists essentially of, or consists of at least 95% by weight ethyl eicosapentaenoate (E-EPA), about 0.2% to about 0.5% by weight ethyl octadecatetraenoate (ODTA-E), about 0.05% to about 0.25% by weight ethyl nonaecapentaenoate (NDPA-E), about 0.2% to about 0.45% by weight ethyl arachidonate (AA-E), about 0.3% to about 0.5% by weight ethyl eicosatetraenoate (ETA-E), and about 0.05% to about 0.32% ethyl heneicosapentaenoate (HPA-E). In another embodiment, the composition is present in a capsule shell. In still another embodiment, the capsule shell contains no chemically modified gelatin.

In another embodiment, compositions useful in accordance with the disclosure comprise, consist essentially of, or consist of at least 95%, 96%, or 97%, by weight, ethyl eicosapentaenoate, about 0.2% to about 0.5% by weight ethyl octadecatetraenoate, about 0.05% to about 0.25% by weight ethyl nonaecapentaenoate, about 0.2% to about 0.45% by weight ethyl arachidonate, about 0.3% to about 0.5% by weight ethyl eicosatetraenoate, and about 0.05% to about 0.32% by weight ethyl heneicosapentaenoate. Optionally, the composition contains not more than about 0.06%, about 0.05%, or about 0.04%, by weight, DHA or derivative thereof such as ethyl-DHA. In one embodiment, the composition contains substantially no or no amount of DHA or derivative thereof such as ethyl-DHA. The composition further optionally comprises one or more antioxidants (e.g., tocopherol) in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell. In another embodiment, the capsule shell contains no chemically modified gelatin.

In another embodiment, compositions useful in accordance with the disclosure comprise, consist essentially of, or consist of at least 96% by weight ethyl eicosapentaenoate, about 0.22% to about 0.4% by weight ethyl octadecatetraenoate, about 0.075% to about 0.20% by weight ethyl nonaecapentaenoate, about 0.25% to about 0.40% by weight ethyl arachidonate, about 0.3% to about 0.4% by weight ethyl eicosatetraenoate and about 0.075% to about 0.25% by weight ethyl heneicosapentaenoate. Optionally, the composition contains not more than about 0.06%, about 0.05%, or about 0.04%, by weight, DHA or derivative thereof such as ethyl-DHA. In one embodiment, the composition contains substantially no or no amount of DHA or derivative thereof such as ethyl-DHA. The composition further optionally comprises one or more antioxidants (e.g., tocopherol) in an amount of not more than about 0.5% or not more than 0.05%. In another embodiment, the composition comprises about 0.05% to about 0.4%, for example about 0.2% by weight tocopherol. In another embodiment, the disclosure provides a dosage form comprising about 500 mg to about 1 g of the foregoing composition in a capsule shell. In one embodiment, the dosage form is a gel- or liquid-containing capsule and is packaged in blister packages of about 1 to about 20 capsules per sheet.

In another embodiment, compositions useful in accordance with the disclosure comprise, consist essentially of, or consist of at least 96%, 97%, or 98%, by weight, ethyl eicosapentaenoate, about 0.25% to about 0.38% by weight ethyl octadecatetraenoate, about 0.10% to about 0.15% by weight ethyl nonaecapentaenoate, about 0.25% to about 0.35% by weight ethyl arachidonate, about 0.31% to about 0.38% by weight ethyl eicosatetraenoate, and about 0.08% to about 0.20% by weight ethyl heneicosapentaenoate. Optionally, the composition contains not more than about 0.06%, about 0.05%, or about 0.04%, by weight, DHA or derivative thereof such as ethyl-DHA. In one embodiment, the composition contains substantially no or no amount of DHA or derivative thereof such as ethyl-DHA. The composition further optionally comprises one or more antioxidants (e.g., tocopherol) in an amount of not more than about 0.5% or not more than 0.05%. In another embodiment, the composition comprises about 0.05% to about 0.4%, for example about 0.2% by weight tocopherol. In another embodiment, the disclosure provides a dosage form comprising about 500 mg to about 1 g of the foregoing composition in a capsule shell. In another embodiment, the capsule shell contains no chemically modified gelatin.

In another embodiment, a composition as described herein is administered to a subject once or twice per day. In another embodiment, 1, 2, 3, or 4 capsules, each containing about 1 g of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 pm and about 11 pm.

In one embodiment, a subject being treated in accordance with methods of the disclosure is not on fibrate or nitrate therapy.

In another embodiment, compositions useful in accordance with methods of the disclosure are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4, or 1 to 2) of times per day, or as many times as needed to elicit a therapeutic response.

In another embodiment, the disclosure provides use of any composition described herein for treating moderate to severe hypertriglyceridemia in a subject in need thereof, comprising: providing a subject having a fasting baseline triglyceride level of about 500 mg/dL to about 1500 mg/dL and administering to the subject a pharmaceutical composition as described herein. In one embodiment, the composition comprises about 1 g to about 4 g of eicosapentaenoic acid ethyl ester, wherein the composition contains substantially no docosahexaenoic acid.

### EXAMPLES

### Example 1

### Safety and Efficacy of Ultra-Pure EPA

A multi-center, placebo-controlled, randomized, double-blind, 12-week study is performed to evaluate the efficacy and safety of >96% E-EPA in patients with fasting triglyceride levels ≥200 mg/dL and <500 mg/dL despite statin therapy (the mean of two qualifying entry values needs to be ≥185 mg/dL and at least one of the values needs to be ≥200 mg/dL). The primary objective of the study is to determine the efficacy of >96% E-EPA 2 g daily and 4 g daily, compared to placebo, in lowering fasting TG levels in patients with high risk for cardiovascular disease and with fasting TG levels ≥200 mg/dL and <500 mg/dL, despite treatment to LDL-C goal on statin therapy.

The secondary objectives of this study are the following:
1. To determine the safety and tolerability of >96% E-EPA 2 g daily and 4 g daily;
2. To determine the effect of >96% E-EPA on lipid and apolipoprotein profiles including total cholesterol (TC), non-high-density lipoprotein cholesterol (non-HDL-C), low density lipoprotein cholesterol (LDL-C), high density lipoprotein cholesterol (HDL-C), and very high density lipoprotein cholesterol (vHDL-C);
3. To determine the effect of >96% E-EPA on lipoprotein associated phospholipase A₂ (Lp-PLA₂) from baseline to week 12;
4. To determine the effect of >96% E-EPA on low-density lipoprotein (LDL) particle number and size;
5. To determine the effect of >96% E-EPA on oxidized LDL;
6. To determine the effect of >96% E-EPA on fasting plasma glucose (FPG) and hemoglobin A_{1c} (HbA_{1c});
7. To determine the effect of >96% E-EPA on insulin resistance;
8. To determine the effect of >96% E-EPA on high-sensitivity C-reactive protein (hsCRP);
9. To determine the effects of >96% E-EPA 2 g daily and 4 g daily on the incorporation of fatty acids into red blood cell membranes and into plasma phospholipids;
10. To explore the relationship between baseline fasting TG levels and the reduction in fasting TG levels; and
11. To explore the relationship between changes of fatty acid concentrations in plasma and red blood cell membranes, and the reduction in fasting TG levels.

The population for this study is men and women >18 years of age with a body mass index ≤45 kg/m² with fasting TG levels greater than or equal to 200 mg/dL and less than 500 mg/dL and on a stable dose of statin therapy (with or without ezetimibe). The statin must be atorvastatin, rosuvastatin, or simvastatin. The dose of statin must be stable for ≥4 weeks prior to the LDL-C/TG baseline qualifying measurement for randomization. The statin dose will be optimal such that the patients are at their LDL-C goal at the LDL-C/TG baseline qualifying measurements. The same statin at the same dose will be continued until the study ends.

Patients taking any additional non-statin, lipid-altering medications (niacin >200 mg/day, fibrates, fish oil, other products containing omega-3 fatty acids, or other herbal products or dietary supplements with potential lipid-altering effects), either alone or in combination with statin therapy (with or without ezetimibe), must be able to safely discontinue non-statin, lipid-altering therapy at screening.

Patients at high risk for CVD, i.e., patients with clinical coronary heart disease (CHD) or clinical CHD risk equivalents (10-year risk >20%) as defined in the National Cholesterol Education Program (NCEP) Adult Treatment Panel III (ATP III) Guidelines will be eligible to participate in this study. Those include patients with any of the following criteria: (1) Known CVD, either clinical coronary heart disease (CHD), symptomatic carotid artery disease (CAD), peripheral artery disease (PAD), or abdominal aortic aneurism; or (2) Diabetes Mellitus (Type 1 or 2).

Approximately 648 patients will be randomized at approximately 80 centers in the U.S. The study will be a 18- to 20-week, Phase 3, multi-center study consisting of 2 study periods: (1) A 6- to 8-week screening period that includes a diet and lifestyle stabilization, a non-statin lipid-altering treatment washout, and an LDL-C and TG qualifying period and (2) A 12-week, double-blind, randomized, placebo-controlled treatment period.

During the screening period and double-blind treatment period, all visits are to be within ±3 days of the scheduled time. All patients will continue to take the statin product (with or without ezetimibe) at the same dose they were taking at screening throughout their participation in the study.

The 6- to 8-week screening period includes a diet and lifestyle stabilization, a non-statin lipid-altering treatment washout, and an LDL-C and TG qualifying period. The screening visit (Visit 1) will occur for all patients at either 6 weeks (for patients on stable statin therapy with or without ezetimibe at screening) or 8 weeks (for patients who will require washout of their current non-statin lipid-altering therapy at screening) before randomization, as follows:
Patients who do not require a washout: The screening visit will occur at Visit 1 (Week −6). Eligible patients will enter a 4-week diet and lifestyle stabilization period. At the screening visit, all patients will receive counseling regarding the importance of the National Cholesterol Education Program (NCEP) Therapeutic Lifestyle Changes (TLC) diet and will receive basic instructions on how to follow this diet.
Patients who will require a washout: The screening visit will occur at Visit 1 (Week −8). Eligible patients will begin a 6-week washout period at the screening visit (i.e. 6 weeks washout before the first LDL-C/TG qualifying visit). Patients will receive counseling regarding the NCEP TLC diet and will receive basic instructions on how to follow this diet. Site personnel will contact patients who do not qualify for participation based on screening laboratory test results to instruct them to resume their prior lipid-altering medications.

Patients having a high risk of cardiovascular disease had a history of coronary artery disease (e.g., history of myocardial infarction, unstable or stable angina, coronary artery interventions, or clinically significant myocardial ischemia), noncoronary forms of clinical atherosclerosis (e.g., peripheral arterial disease, abdominal aortic aneurysm, or carotid artery disease), or type 1 or 2 diabetes mellitus. Patients were excluded from the study if they had received peritoneal dialysis or hemodialysis for renal insufficiency. Patients were randomly allocated to receive 4 g/day E-EPA, 2 g/day E-EPA, or placebo. Exclusion criteria in the ANCHOR study related to renal function included known nephrotic range (greater than 3 g/day) proteinuria, history or evidence of major and clinically significant renal disease that would interfere with the conduct of the study or interpretation of the data, and requirement for peritoneal dialysis or hemodialysis for renal insufficiency.

At the end of the 4-week diet and lifestyle stabilization period or the 6-week diet and stabilization and washout period, eligible patients will enter the 2-week LDL-C and TG qualifying period and will have their fasting LDL-C and TG levels measured at Visit 2 (Week -2) and Visit 3 (Week -1). Eligible patients must have an average fasting LDL-C level ≥40 mg/dL and <100 mg/dL and an average fasting TG level ≥200 mg/dL and <500 mg/dL to enter the 12-week double-blind treatment period. The LDL-C and TG levels for qualification will be based on the average (arithmetic mean) of the Visit 2 (Week -2) and Visit 3 (Week -1) values. If a patient's average LDL-C and/or TG levels from Visit 2 and Visit 3 fall outside the required range for entry into the study, an additional fasting lipid profile can be collected 1 week later at Visit 3.1. If a third sample is collected at Visit 3.1, entry into the study will be based on the average (arithmetic mean) of the values from Visit 3 and Visit 3.1.

After confirmation of qualifying fasting LDL-C and TG values, eligible patients will enter a 12-week, randomized, double-blind treatment period. At Visit 4 (Week 0), patients will be randomly assigned to 1 of the following treatment groups:
>96% E-EPA 2 g daily,
>96% E-EPA 4 g daily, or
Placebo.

Approximately 216 patients per treatment group will be randomized in this study. Stratification will be by type of statin (atorvastatin, rosuvastatin, or simvastatin), the presence of diabetes, and gender.

During the double-blind treatment period, patients will return to the site at Visit 5 (Week 4), Visit 6 (Week 11), and Visit 7 (Week 12) for efficacy and safety evaluations.

Eligible patients will be randomly assigned at Visit 4 (Week 0) to receive orally >96% E-EPA 2 g daily, >96% E-EPA 4 g daily, or placebo.

>96% E-EPA is provided in 1 g liquid-filled, oblong, gelatin capsules. The matching placebo capsule is filled with light liquid paraffin and contains 0 g of >96% E-EPA. >96% E-EPA capsules are to be taken with food (i.e. with or at the end of a meal).

During the double-blind treatment period, patients will take 2 capsules (>96% E-EPA or matching placebo) in the morning and 2 capsules in the evening for a total of 4 capsules per day.
Patients in the >96% E-EPA 2 g/day treatment group will receive 1>96% E-EPA 1 g capsule and 1 matching placebo capsule in the morning and in the evening.
Patients in the >96% E-EPA 4 g/day treatment group will receive 2>96% E-EPA 1 g capsules in the morning and evening.

Patients in the placebo group will receive 2 matching placebo capsules in the morning and evening.

The primary efficacy variable for the double-blind treatment period is percent change in TG from baseline to Week 12 endpoint. The secondary efficacy variables for the double-blind treatment period include the following:
Percent changes in total cholesterol (TC), high-density lipoprotein cholesterol (HDL-C), LDL-C, calculated non-HDL-C, and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A-I/apo B ratio from baseline to Week 12;
Percent changes in lipoprotein(a) from baseline to Week 12;
Percent changes in LDL particle number and size, measured by nuclear magnetic resonance, from baseline to Week 12;
Percent change in remnant-like particle cholesterol from baseline to Week 12;
Percent change in oxidized LDL from baseline to Week 12;
Changes in FPG and HbA_{1c} from baseline to Week 12;
Change in insulin resistance, as assessed by the homeostasis model index insulin resistance, from baseline to Week 12;
Percent change in lipoprotein associated phospholipase A₂ (Lp-PLA₂) from baseline to Week 12;
Change in intracellular adhesion molecule-1 from baseline to Week 12;
Change in interleukin-2 from baseline to Week 12;
Change in plasminogen activator inhibitor-1 from baseline to Week 12. Note: this parameter will only be collected at sites with proper storage conditions;
Change in hsCRP from baseline to Week 12; and
Change in plasma concentration and red blood cell membrane content of fatty acid from baseline to Week 12 including EPA, docosapentaenoic acid (DPA), docosahexaenoic acid (DHA), arachidonic acid (AA), dihomo-γ-linolenic acid (DGLA), the ratio of EPA/AA, ratio of oleic acid/stearic acid (OA/SA), and the ratio of total omega-3 acids over total omega-6 acids.

Safety assessments will include adverse events, clinical laboratory measurements (chemistry, hematology, and urinalysis), 12-lead electrocardiograms (ECGs), vital signs, and physical examinations.

For TG, TC, HDL-C, LDL-C, calculated non-HDL-C, and VLDL-C, baseline will be defined as the average of Visit 4 (Week 0) and the preceding lipid qualifying visit (either Visit 3 [Week -1] or if it occurs, Visit 3.1) measurements. Baseline for all other efficacy parameters will be the Visit 4 (Week 0) measurement.

For TG, TC, HDL-C, LDL-C, calculated non-HDL-C, and VLDL-C, Week 12 endpoint will be defined as the average of Visit 6 (Week 11) and Visit 7 (Week 12) measurements.

Week 12 endpoint for all other efficacy parameters will be the Visit 7 (Week 12) measurement.

The primary efficacy analysis will be performed using a 2-way analysis of covariance (ANCOVA) model with treatment as a factor and baseline TG value as a covariate. The least-squares mean, standard error, and 2-tailed 95% confidence interval for each treatment group and for each comparison will be estimated. The same 2-way ANCOVA model will be used for the analysis of secondary efficacy variables.

The primary analysis will be repeated for the per-protocol population to confirm the robustness of the results for the intent-to-treat population.

Non-inferiority tests for percent change from baseline in LDL-C will be performed between >96% E-EPA doses and placebo using a non-inferiority margin of 6% and a significant level at 0.05.

For the following key secondary efficacy parameters, treatment groups will be compared using Dunnett' s test to control the Type 1 error rate: TC, LDL-C, HDL-C, non-HDL-C, HDL-C, Lp-PLA₂, and Apo B. For the remaining secondary efficacy parameters, Dunnett' s test will not be used and the ANCOVA output will be considered descriptive.

The evaluation of safety will be based primarily on the frequency of adverse events, clinical laboratory assessments, vital signs, and 12-lead ECGs. The primary efficacy variable is the percent change in fasting TG levels from baseline to Week 12. A sample size of 194 completed patients per treatment group will provide 90.6% power to detect a difference of 15% between >96% E-EPA and placebo in percent change from baseline in fasting TG levels, assuming a standard deviation of 45% in TG measurements and a significance level of p<0.05.

Previous data on fasting LDL-C show a difference in percent change from baseline of 2.2%, with a standard deviation of 15%, between study drug and placebo. A sample size of 194 completed patients per treatment group will provide 80% power to demonstrate non-inferiority (p <0.05, one-sided) of the LDL-C response between >96% E-EPA 4 g daily and placebo, within a 6% margin. To accommodate a 10% drop-out rate from randomization to completion of the double-blind treatment period, a total of 648 randomized patients is planned (216 patients per treatment group).

### Example 2

### EPA Treatment of Patients Exposed to Polluted Air and/or Particulate Matter

Exposure to air pollution is associated with an increased risk for oxidative stress, endothelial dysfunction, narrowed and/or thickened arteries, and/or inflammation. Polluted air frequently contains particulate matter that contributes to the pathogenesis of cardiovascular disease.

Human exposure studies will be conducted to investigate the effects of icosapent ethyl (E-EPA) to treat and prevent the adverse effects of exposure to air pollution. This study will include an assessment on the efficacy of E-EPA to treat and prevent oxidative stress, endothelial dysfunction, narrowing and/or thickening arteries and/or inflammation on patients exposed to both long term (i.e., exposures of a year or more) and short term (i.e., exposures of less than one year) air pollution. The study will also include a subset of patients who have not been exposed to air pollution. These patients will be administered E-EPA and will then be exposed to polluted air with a known concentration of particulate matter to assess the ability to E-EPA to prevent the adverse effects associated with exposure to air pollution. Patients will be randomized into to two cohorts: (1) patients randomized to E-EPA and (2) patients randomized to a placebo control. Provide below is a table of the two cohorts and the three patient populations defined by the duration of exposure to air pollution prior to initiating therapy.

### Treatment Groups:

| Cohort | Patient Group 1 | Patient Group 2 | Patient Group 3 |
|---|---|---|---|
| (1) 4 g/day | long term exposure | short term exposure | no prior exposure |
| E-EPA | | | |
| (2) placebo | long term exposure | short term exposure | no prior exposure |

The primary objective of this study will be to determine if and how 4 g of daily of E-EPA, compared to placebo, affects patients exposed to polluted air. In particular, an assessment on the effects of E-EPA on the patient's inflammatory biomarkers, metabolic biomarkers, oxidation biomarkers, changes in composition of the arterial wall (including changes plaque characteristics), and beneficial effects of heart rate rhythm will be conducted. The secondary objective is to determine if and how 4 g daily E-EPA, compared to placebo, affects CVD-related parameters in patients exposed to air pollution and having a high risk for CVD.

Inflammatory biomarkers will include TNFα, MCP-1, IL-1β, sICAM-1, sVCAM-1, hsCRP, Lp-PLA₂, circulating monocytes, IL-6, or any combination thereof

Metabolic biomarkers will include TC, VLDL-C, LDL-C, HDL-C, non-HDL-C, Apo B, Apo A-1, HDL-C functionality, HOMA-IR levels, or any combination thereof.

Oxidative biomarkers will include lipid oxidation, lipid peroxidation, lipid hydroperoxidation, malondialdehyde, PGF-2α, PDGF, antioxidant potential levels, or any combination thereof.

Beneficial effects in heart rate rhythm will include an assessment on arrhythmia suppression, ventricular arrhythmia rate, HRV, heart rate, and any combination thereof.

These CVD-parameters include lipid levels, lipoprotein levels, and inflammatory markers, such as TC, LDL-C, HDL-C, VLDL-C, VLDL-TG, RLP-C, non-HDL-C, Apo B, Apo C-III, Lp-PLA, hsCRP, and ox-LDL, in plasma and RBC EPA concentration, or any combination thereof.

Measurements of the patient's inflammatory biomarkers, metabolic biomarkers, heart rate, and CVD-parameters will be determined prior to and after administration of the E-EPA. The median differences in the inflammatory biomarkers, metabolic biomarkers, changes in heart rate, and CVD-parameters will be determined from baseline to after administration of E-EPA compared to placebo.

1. A method of treating or preventing oxidative stress, endothelial dysfunction, narrowing and/or thickening of arteries, and/or inflammation induced by inhalation of particulate matter in a subject, the method comprising administering to the subject a composition comprising about 4 g of eicosapentaenoic acid per day.

2. The method of claim 1, wherein the particulate matter is less than about 10 μm and greater than about 2.5 μm in diameter.

3. The method of claim 1, wherein the particulate matter is less than or equal to about 2.5 μm in diameter.

4. The method of claim 1, where in the particulate matter is less than about 0.1 μm in diameter.

5. The method of claim 1, wherein the inflammation is pulmonary and/or systemic inflammation.

6. The method of claim 1, wherein administration of the composition reduces a risk of atherosclerotic cardiovascular disease in the subject.

7. The method of claim 1, wherein the subject experiences a reduction in blood pressure levels.

8. The method of claim 1, wherein the subject experiences a reduction in insulin resistance.

9. The method of claim 1, wherein the subject experiences a reduction in an inflammatory biomarker selected from the group consisting of vascular endothelial growth factor (VEGF), tumor necrosis factor-α (TNF-α), monocyte chemoattractant protein-1 (MCP-1), interleukin-1β (IL-1β), soluble intercellular adhesion molecule-1 (sICAM-1), soluble vascular cellular adhesion molecule-1 (sVCAM-1), high sensitivity reactive protein (hsCRP), lipoprotein-associated phospholipase A₂ (Lp-PLA₂), and circulating monocyte levels.

10. The method of claim 1, wherein the subject experiences a reduction in a metabolic biomarker selected from the group consisting of total cholesterol (TC), very low dense lipoprotein cholesterol (VLDL-C), low dense lipoprotein cholesterol (LDL-C), high dense lipoprotein cholesterol (HDL-C), non-high dense lipoprotein cholesterol (non-HDL-C), HDL-C functionality, apolipoprotein B (Apo B), interleukin-6 (IL-6), apolipoprotein A-1 (Apo A-1), and homeostasis model assessment of insulin resistance (HOMA-IR) levels.

11. The method of claim 1, wherein the subject experiences a reduction in an oxidative biomarker selected from the group consisting of lipid oxidation, lipid peroxidation, lipid hydroperoxidation, malondialdehyde, prostaglandin-2α (PGF-2α), platelet-derived growth factor (PDGF), and antioxidant potential levels.

12. The method of claim 1, wherein the subject exhibits beneficial effects in heart rate and/or rhythm following administration of the composition.

13. The method of claim 12, wherein the beneficial effects include a reduction in arrhythmia suppression levels, ventricular arrhythmia rates, or heart rate or an increase in heart rate variability.

14. The method of claim 1, wherein the composition is administered to the subject in 1 to 4 dosage units per day.

15. The method of claim 1, wherein the eicosapentaenoic acid comprises at least about 96 wt.% of all omega-3 fatty acids in the composition.

16. A method of treating or preventing oxidative stress, endothelial dysfunction, narrowing and/or thickening of arteries, and/or inflammation induced by long term and/or short term exposure to air pollution in a subject, the method comprising administering to the subject a composition comprising about 4 g of eicosapentaenoic acid per day.

17. The method of claim 16, wherein the air pollution contains particulate matter.

18. The method of claim 16, wherein the particulate matter is less than about 10 μm and greater than about 2.5 μm in diameter.

19. The method of claim 16, wherein the particulate matter is less than or equal to about 2.5 μm in diameter.

20. The method of claim 16, where in the particulate matter is less than about 0.1 μm in diameter.

21. The method of claim 16, wherein the inflammation is pulmonary and/or systemic inflammation.

22. The method of claim 16, wherein administration of the composition reduces a risk of atherosclerotic cardiovascular disease in the subject.

23. The method of claim 16, wherein the subject experiences a reduction in blood pressure levels.

24. The method of claim 16, wherein the subject experiences a reduction in insulin resistance.

25. The method of claim 16, wherein the subject experiences a reduction in an inflammatory biomarker selected from the group consisting of vascular endothelial growth factor (VEGF), tumor necrosis factor-α (TNF-α), monocyte chemoattractant protein-1 (MCP-1), interleukin-1β (IL-1β), soluble intercellular adhesion molecule-1 (sICAM-1), soluble vascular cellular adhesion molecule-1 (sVCAM-1), high sensitivity reactive protein (hsCRP), lipoprotein-associated phospholipase A₂ (Lp-PLA₂), and circulating monocyte levels.

26. The method of claim 16, wherein the subject experiences a reduction in a metabolic biomarker selected from the group consisting of total cholesterol (TC), very low dense lipoprotein cholesterol (VLDL-C), low dense lipoprotein cholesterol (LDL-C), high dense lipoprotein cholesterol (HDL-C), non-high dense lipoprotein cholesterol (non-HDL-C), HDL-C functionality, apolipoprotein B (Apo B), interleukin-6 (IL-6), apolipoprotein A-1 (Apo A-1), and homeostasis model assessment of insulin resistance (HOMA-IR) levels.

27. The method of claim 16, wherein the subject experiences a reduction in an oxidative biomarker selected from the group consisting of lipid oxidation, lipid peroxidation, lipid hydroperoxidation, malondialdehyde, prostaglandin-2α (PGF-2α), platelet-derived growth factor (PDGF), and antioxidant potential levels.

28. The method of claim 16, wherein the subject exhibits beneficial effects in heart rate and/or rhythm following administration of the composition.

29. The method of claim 28, wherein the beneficial effects include a reduction in arrhythmia suppression levels, ventricular arrhythmia rates, or heart rate or an increase in heart rate variability.

30. The method of claim 16, wherein the composition is administered to the subject in 1 to 4 dosage units per day.

31. The method of claim 16, wherein the eicosapentaenoic acid comprises at least about 96 wt.% of all omega-3 fatty acids in the composition.

32. A method of treating or preventing oxidative stress, endothelial dysfunction, narrowing and/or thickening of arteries, and/or inflammation induced by inhalation of particulate matter in a subject, the method comprising administering to the subject a composition comprising about 4 g of eicosapentaenoic acid per day, wherein administration of the composition reduces a risk of atherosclerotic cardiovascular disease in the subject.

33. The method of claim 32, wherein the air pollution contains particulate matter.

34. The method of claim 32, wherein the particulate matter is less than about 10 μm and greater than about 2.5 μm in diameter.

35. The method of claim 32, wherein the particulate matter is less than or equal to about 2.5 μm in diameter.

36. The method of claim 32, where in the particulate matter is less than about 0.1 μm in diameter.

37. The method of claim 32, wherein the inflammation is pulmonary and/or systemic inflammation.

38. The method of claim 32, wherein the subject experiences a reduction in blood pressure levels.

39. The method of claim 32, wherein the subject experiences a reduction in insulin resistance.

40. The method of claim 32, wherein the subject experiences a reduction in an inflammatory biomarker selected from the group consisting of vascular endothelial growth factor (VEGF), tumor necrosis factor-α (TNF-α), monocyte chemoattractant protein-1 (MCP-1), interleukin-1β (IL-1β), soluble intercellular adhesion molecule-1 (sICAM-1), soluble vascular cellular adhesion molecule-1 (sVCAM-1), high sensitivity reactive protein (hsCRP), lipoprotein-associated phospholipase A₂ (Lp-PLA₂), and circulating monocyte levels.

41. The method of claim 32, wherein the subject experiences a reduction in metabolic biomarker selected from the group consisting of total cholesterol (TC), very low dense lipoprotein cholesterol (VLDL-C), low dense lipoprotein cholesterol (LDL-C), high dense lipoprotein cholesterol (HDL-C), non-high dense lipoprotein cholesterol (non-HDL-C), HDL-C functionality, apolipoprotein B (Apo B), interleukin-6 (IL-6), apolipoprotein A-1 (Apo A-1), and homeostasis model assessment of insulin resistance (HOMA-IR) levels.

42. The method of claim 32, wherein the subject experiences a reduction in an oxidative biomarker selected from the group consisting of lipid oxidation, lipid peroxidation, lipid hydroperoxidation, malondialdehyde, prostaglandin-2α (PGF-2α), platelet-derived growth factor (PDGF), and antioxidant potential levels.

43. The method of claim 32, wherein the subject exhibits beneficial effects in heart rate and/or rhythm following administration of the composition.

44. The method of claim 43, wherein the beneficial effects include a reduction in arrhythmia suppression levels, ventricular arrhythmia rates, or heart rate or an increase in heart rate variability.

45. The method of claim 32, wherein the composition is administered to the subject in 1 to 4 dosage units per day.

46. The method of claim 32, wherein the eicosapentaenoic acid comprises at least about 96 wt.% of all omega-3 fatty acids in the composition.
